(19) United States
(12) Patent Application Publication
(10) Pub. No.: US 2009/0187232 A1
(43) Pub. Date: Jul. 23, 2009

(54) Systems And Methods For Therapeutic Treatments

(76) Inventor: Issa Salim, Dubai (AE)

Correspondence Address: KILPATRICK STOCKTON LLP, 1001 WEST FOURTH STREET, WINSTON-SALEM, NC, 27101, US

(21) Appl. No.: 12/355,194

(22) Filed: Jan. 16, 2009

Related U.S. Application Data
(60) Provisional application No. 61/021,375, filed on Jan. 16, 2008.

(51) Int. Cl. A61N 1/04; A61B 5/05; G09B 19/00

(52) U.S. Cl. 607/46; 600/407; 434/127

(57) ABSTRACT

The present invention relates to systems and methods to diagnose and then provide therapy and/or treating humans to relieve or reduce the symptoms of various disease and/or conditions, and in certain embodiments reduce the factors causing the symptoms and/or diseases. The present invention also relates to systems and methods for determining heretofore unknown internal characteristics of patients and external environmental factors.

### CROSS-REFERENCES TO RELATED APPLICATION

This application claims priority to U.S. Provisional Patent Application No. 61/021,375 entitled “Systems And Methods For Therapeutic Treatments,” filed Jan. 16, 2008, the entirety of which is incorporated herein by reference.

### FIELD OF THE INVENTION

The present invention relates to systems and methods for providing therapy and/or treating humans to relieve or reduce the symptoms of various diseases and/or conditions, and in certain embodiments reduce the factors causing the symptoms and/or diseases. The present invention also relates to systems and methods for determining the causes of diseases and/or symptoms. The present invention further relates to determining heretofore unknown internal characteristics of patients and external environmental factors.

### BACKGROUND OF THE INVENTION

Although the state of medicine and medicinal science is well advanced, there remain numerous conditions that cause pain and suffering in individuals that are incapable of diagnosis and/or treatment by heretofore known medical techniques and medicines. Often, patients suffering with such conditions are offered medications that treat the symptoms of the pain without treating the underlying cause of the pain. In certain cases, pain medications themselves may not be adequate to remove a patient's pain and suffering.

Bioresonance therapy was first invented in 1977 by the German scientists Franz Morell and Erich Rasche. However, to date, bioresonance therapy remains poorly understood. The German physicist Burkhard Heim founded a Unified Quantum Field theory that attempted to explain, in physical and mathematical terms, those biological processes in an organism which had been observed, but for which no satisfactory theoretical explanation had yet been given. Heim's theories have important consequences for medicine by opening up an understanding of additional dimensions beyond the conventional three dimensional approach to medicine. A new approach that utilizes bioresonance therapy could benefit patients and mankind.

Thus, there is a need for systems and methods that provide accurate diagnosis and/or treatments for patients suffering from various diseases and/or conditions. There is also a need for systems and methods to determine the causes of various diseases and/or conditions. Further, there is a need to determine heretofore unknown internal characteristics of patients and external environmental factors as they relate to the pain and suffering of individuals.

### SUMMARY OF THE INVENTION

In some embodiments, the present invention provides systems and methods for determining the causes of pain, suffering and/or discomfort being experienced by a patient. In some other embodiments, the present invention systems and methods for treating the causes of pain, suffering and/or discomfort. In some other embodiments, the present invention provides systems and methods for treating pain, suffering and/or discomfort. In other embodiments, the present invention provides systems and methods for determining the cause of pain, suffering and/or discomfort being experienced by a patient, treating the cause of the pain, suffering and/or discomfort, and treating the pain, suffering and/or discomfort. In further embodiments, the present invention provides systems and methods for determining heretofore unknown internal characteristics of patients and external environmental factors. Such characteristics may be useful in patient care.

The conceptual basis for some embodiments of the present invention comprises bioresonance therapy. Bioresonance relates to the interaction between and among matter and external fields, such as electromagnetic fields. Bioresonance therapy, as utilized in embodiments of the present invention, utilizes the interaction in the diagnosis and/or treatment of patients. A more detailed description of bioresonance, and the systems and methods of the present invention is set forth below with reference to the appended figures.

### BRIEF DESCRIPTION OF THE DRAWINGS

FIG. 1 is a schematic diagram illustrating the relationship between space, time and energy.

FIG. 2 is a diagram illustrating the interaction between matter, energy and fields.

FIG. 3 is a diagram illustrating the frequency hertz for various types of rays.

FIG. 4 is an illustration of different dimensions in which diagnoses can be made.

FIG. 5 is a representative readout of an Arterial Brachial Pressure Index before and after bioresonance therapy.

FIG. 6 is a blood film from a hypothyroidism patient. The arrows indicate the presence of white blood cells.

### DETAILED DESCRIPTION OF THE INVENTION

The present invention provides systems and methods for providing a diagnosis of the causes of diseases and/or symptoms, therapy and/or treatment to relieve or reduce symptoms of various diseases and/or symptoms, treatment of pain related to various diseases and/or symptoms, and systems and methods for determining heretofore unknown internal characteristics of patients and external environmental factors. Embodiments of the present invention are useful for the diagnosis, treatment, and prevention of diseases and/or symptoms in an individual.

An aspect of the present invention relates to patient diagnosis. In some embodiments, the present invention comprises a method for diagnosing a disease and/or condition in a patient. The method for diagnosis may comprise the steps of: exposing at least a portion of a patient to an external field, determining a bioresonance; and correlating the bioresonance with bioresonances of a disease state or symptom.

Another aspect of the present invention relates to the treatment of patients to reduce pain, suffering, discomfort and/or other symptoms of disease states. In another embodiment of the present invention, the cause of the pain, suffering, discomfort and/or other symptoms may be determined by a method or system of the present invention, or may be determined in other ways, or known. In some embodiments, a method may comprise: exposing at least a portion of a patient to an external field to affect the bioresonance of the portion of the patient, or a pathogen.

Another aspect of the present invention relates to treatment of the underlying causes of a patient's pain, suffering and/or discomfort. The cause of the pain, suffering, discomfort and/or other symptoms may be determined by a method or system of the present invention, or may be determined in other ways, or known. In some embodiments, a method may comprise: exposing at least a portion of a cause of a patient's pain, suffering and/or discomfort to an external field to affect the bioresonance of the cause.

In some embodiments relating to the foregoing aspects of the present invention, the external field comprises a field sufficient to generate a bioresonance, including, but not limited to an electromagnetic field. Certain patients may have pre-existing medical conditions, and/or implanted medical devices (e.g. a pacemaker) that may be adversely affected by an external field. Thus, in some embodiments of the present invention, prescreening patients, for example through a medical questionnaire, or a review of a patient's medical history to determine whether the the patient is suitable for exposure to an external field and aid in the choice of external field.

Some embodiments of the present invention use a holistic approach to therapies. In such embodiments, a bioresonance of multiple portions of a patient are determined.

In another aspect, the present invention relates to determining a characteristic of a portion of a patient. In some embodiments a method comprises: exposing a portion of a patient to an external field and determining the bioresonance of the portion. As used herein, portion refers to organs; tissues; fluids; glands; nerves; bones; systems and portions therefore (e.g. hormonal systems) and any other discrete parts of a patient. In some embodiments, a method of the present invention comprises establishing a medical history for a patient, the medical history comprising a bioresonance of a portion of the patient, in some embodiments a bioresonance of multiple portions of a patient. These “historic” bioresonances may be utilized as a diagnostic tool through a comparison between/among a new bioresonance measurement and an historic bioresonance measurement.

In another aspect, the present invention relates to determining a characteristic of an external factor. An external factor refers to a factor other than a portion of a patient. In some embodiments a method may comprise: exposing an external factor to an external field and determining the bioresonance of the factor. In some embodiments of the present invention, a bioresonance of an external factor may be utilized as part of a determination of a cause of a patient's pain, suffering and/or discomfort, and/or as an aid in the diagnosis of a disease state, for example by comparing/matching a bioresonance from a portion of a patient to the bioresonance of an external factor.

Thus, some embodiments of methods may include one or more of the following tests, wherein “test” refers to steps, such as those set forth herein, to determine a bioresonance: organ frequency tests; blood frequency tests; tests to determine allergic strains. Tests to determine allergic strains may include tests for allergies to foods (such as wheat, eggs, spices, etc.); food additives (such as flavorings, emulsifiers, colorants, preservatives, sweeteners, thickeners, etc.); inhaled particles (pollen, mildew, mites, animal dander, pesticides, etc.); chemical substances; insects; vaccines (such as MMR, Polio, etc.); heavy metals (aluminum, lead, cadmium, gold, cobalt, copper, nickel, palladium, mercury, silver, etc.); bacteria, parasites, or fungi. Tests may also include tests for viruses, tumors, the presence of known carcinogens. In some embodiments, the test may comprise a test of blood, urine, stool, or other body fluids. In other embodiments, tests may include tests of a patients' personal products, such as shampoo, cosmetics, perfumes, etc. In yet other embodiments, tests may include tests of any medications or other drugs taken by the patient.

Benefits of running and monitoring the foregoing tests as a portion of a therapy program include: enhancing communication between the right and left hemisphere of the brain; enhancing the function of the immune system; enhancing detoxification of the liver; enhancing the function of the endocrine system; managing pain, elimination of parasites; correcting defects in blood frequency; correcting defects in organ frequency; allergen desensitization, etc.

An external field may be generated in embodiments of the present invention and a bioresonance determined. A system of the present invention comprises equipment, including computer equipment, for performing a method of the present invention.

The resonances utilized will vary depending on the treatment therapies. For example, in some embodiments of the present invention, the following resonances may be utilized in the following treatments: prevention of chronic diseases in pediatrics by blood frequency modification of mothers before and during pregnancy: 52 KHz, 110 KHz, 114 KHz, 130 KHz; detecting gynecological diseases by using male blood for diagnosis and treatment 70 Hz, 95 Hz, 53 KHz, 830 Hz, 110 KHz, 128 KHz, 140 KHz; measuring heavy metals in the brain of Autistic children: 52 KHz, 2.5 kHz, 110 KHz, 130 KHz, 10-150 kHz; diagnosis and treatment of retinal defects: 52 KHz, 110 KHz, 114 KHz, 127 KHz, 128 KHz, 20 KHz, 10 Hz, 144 KHz, 450 Hz; detecting local and systemic parasites in blood 52 KHz, 110 KHz, 130 KHz, 10 Hz, 145 KHz. Further details on these embodiments are set forth below.

In some embodiments, the present invention provides systems and methods for preventing chronic diseases in fetuses by blood frequency modification of the mother before and during pregnancy. The present inventor has discovered that chronic diseases (such as eczema, epilepsy, and kidney failure, etc.) in children are highly related to the status of their blood frequency (BF) during pregnancy. Pregnant mothers with defective BF transfer that defect to their fetuses. After delivery, after the child receives his first vaccine, multiple problems arise and continue as the child ages. Medication intake, vitamins, pollution, allergic food substances and other factors accelerate the occurrence of chronic disease and increase the resistance to any type of treatment. On the other hand, pregnant mothers with normal BF give birth to newborns with normal BF. Since the newborn has a normal BF, the newborn is less susceptible to chronic diseases and vaccinations induce no harm on his body.

In some embodiments, the present invention provides methods and systems for detecting gynecological diseases by using male blood for diagnosis and treatment. The present inventor has discovered that gynecological diseases of females may be susceptible to diagnosis and treatment using male's blood only. Blood carries the information of the whole body's various systems and can be used effectively in diagnosis and treatment. By comparison of the frequency of the female patient in the presence or absence of the male blood probe, a determination of the health of the female can be determined.

In some embodiments, the present invention provides systems and method for measuring heavy metals' in the brain of Autistic children. These embodiments are the first, and possibly only, systems and methods for autism diagnosis using frequency measurements and are separate and apart from the usual and traditional methods that evaluate the patient's IQ to determine whether he is autistic or not. A method of the present invention is a very effective and accurate tool that has a very limited range of false results. In these embodiments, the 10/20 system may be set to measure the existence of the heavy metals in the brain of the child to detect not only the existence of the ions but also the intensity of the ions present in the brain.

In some embodiments, the present invention provides systems and methods for diagnosing and treating retinal defects. Optical Coherence Tomography (OCT) is a technique for obtaining sub-surface images of translucent or opaque materials at a resolution equivalent to a low-power microscope. By using light, OCT delivers high resolution images that can be use to generate high resolution three-dimensional images of the retina. However, measuring the thickness of the retina in diabetic patients via OCT is very complicated. An embodiment of the present invention provides systems and methods that may be utilized to detect the intensity of the thickness as well to treat it.

In some embodiments, the present invention provides systems and methods for detecting parasites in blood. Parasite infections could be either systemic or local. Systemic infestation of parasites occurs when they invade the lymphatic system and circulate with the lymphatic vessels all over the body. However, localized infestation occurs when the parasites invade a localized area like the colon, lungs, and uterus and induce their damage on the cells of the infested region only. Localized parasite infestation is usually harder to treat than systemic infestation because the damage induced is more vigorous and the parasite needs a longer time to be eliminated. If the parasite is left untreated, the infected area could become cancerous. An embodiment of the present invention provides methods and systems for treatment of the parasite. The treatment depend on the kind of the parasite, while each parasite has own frequency, however, several potential frequencies are listed above. The role of bioresonance therapy in treating parasitic infections is to detect the location of the parasite, to measure its intensity, to eliminate the parasite, and to then regenerate the organ that was infested. The treatment plan for parasitic infections in bioresonance therapy includes subjecting a patient to several therapy programs that enhance the function of the immune system, the detoxification process of the liver, and mucus secretion (defense barrier) in the colon. To accelerate the process of parasite elimination, the patient should follow a diet free of animal proteins, the major source of nutrients for parasites, for at least one month.

In yet another embodiment of the present invention, systems and methods are provided for treatment of peripheral vascular disease. In a further embodiment, the treatment of peripheral vascular disease is in diabetic patients. Diabetes is a complicated disease implicating multiple organs and factors. Diabetes disrupts the vascular system and causes deleterious effects in the eyes, kidneys, legs, and feet, as well as in other organs and organ systems. Peripheral vascular disease in diabetic patients serves to inhibit the individual's blood circulation, frequently to the extremities. In diabetic patients, there is a narrowing of the arteries that leads to significantly decreased circulation in the lower part of the legs and the feet. Poor circulation contributes to the myriad of foot problems from which diabetic patients suffer by reducing the amount of oxygen and other nutrients that reach the skin and tissues of the feet. Among other things, poor circulation leads to reduced wound healing ability and swelling and/or dryness of the feet. Poor circulation may further lead to ulcers and infections in the feet of diabetic patients. An embodiment of the present invention provides a method for the treatment of poor circulation in diabetic patients.

Embodiments of the present invention should not be limited or viewed as relying on a particular theory for operation. It is believed, however, that the features of embodiments of the present invention may be better understood and appreciated through an understanding of bioresonance as set forth below with reference to certain of the drawings.

A deeper understanding of physics opens better diagnostic and therapeutic possibilities to a medical doctor. Magnet resonance imaging (MRI) for instance, based on the magnetic resonance of atomic nuclei is a diagnostic conversion. The bioresonance therapy, which based on the interaction between matter and electromagnetic fields, is a therapeutic conversion.

Biophysics relates the findings of quantum physics to applications relating to biological systems. As often happens with new scientific developments, biophysics, and bioresonance, have stood in conflict with other lines of thinking. However, this research and further research promises to bring more clarity. Ultimately, improved therapy results are of a higher priority than obtaining a homogenous theory that does not produce the desired results. Bioresonance therapy surely offers the improved therapy results.

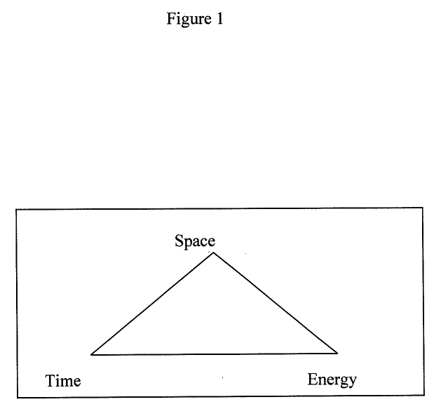

Apart from space and time, the universe comprises energy as represented in FIG. 1. All observations can be derived from the different interactions between these three constituents. From the point of view of physics everything always consists of a combination of these three factors. So there is no empty space, in other words: space without energy. Everywhere there are forces (a way of manifestation of energy) present as for instance gravitation. These three constituents seem inseparable and mutually influencing (interaction), which is why in astrophysics for instance they speak of space and time.

Energy can manifest itself as a particle, as well as a wave in many different forms. But energy is always classified as particle. On the one hand there is matter, a condensed form of energy with the properties of mass. On the other hand there are spaces in which “invisible” energies (forces) prevail that only make themselves noticeable when they find a corresponding medium on which they can take effect. Theses spaces are called fields (by analogy with space energies).

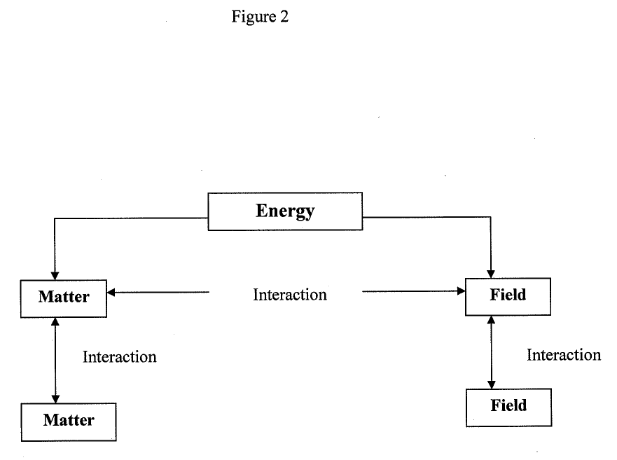

A typical example of a field is the electromagnetic field created by a radio transmitter. This field is not noticeable until a corresponding radio receiver is used. Another everyday example of a field is the field of gravitation of the earth. Fields as well as matter can have an interaction or influence each other mutually. As shown in FIG. 2 there can be interaction between a) matter and matter; b) matter and field; and/or c) field and field.

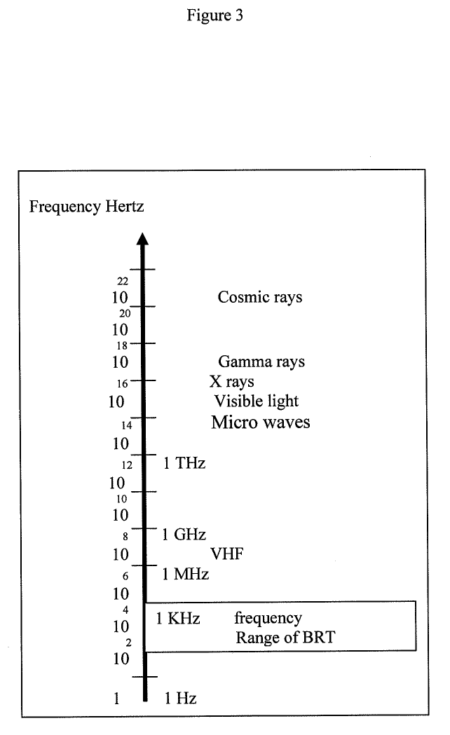

Photons, existing as electromagnetic waves exist in a huge frequency range. An overview is shown in FIG. 3. In general, the higher the frequencies, the higher in energy are rays and the shorter the wavelength.

The matter part of the human body (70 kg) comprise almost 1.4×10²⁹ matter particles. It is assembled from approximately 10 trillion (10¹²) cells. A cell consists of around 20 billion (2×10¹⁰) proteins molecules. A single protein molecule is, on average, built from 14,000 (1.4×10⁴) atoms. One atom, for example nitrogen, is composed of 7 electrons, 7 protons (21 quarks) and 7 neutrons (21 quarks), thus in total 49 matter particles.

However, matter is not the most common form of energy. Carlo Rubbia established that the amount of other forms of energies (protons) strongly predominates the matter particles. The exact ratio is one matter particle against 974,600,000 photons. In the case of the human body, this means that only the smallest part consists of matter (approximately 1.4×10³⁸).

Therefore, the different energetic compositions of a human being that are possible as a consequence of this fact are nearly incomprehensible (10¹⁰⁴⁷) as well as the resulting possible changes in the situation per second: 4×10⁵³.

These forces cause various effects in the movement, structure, properties, process and energy of matter. There are four elementary forces: strong nuclear linkage, weak nuclear linkage, electromagnetic force, and gravitation. All other kinds of forces (wind, friction, centrifugal force) stem from these elementary forces.

Strong nuclear linkage is the strongest force known. Electromagnetic force is second strongest force in the universe and is only one hundredth (10⁻²) of the force of a strong nuclear linkage. A weak nuclear linkage is only one ten-thousands (10⁻⁵) of a strong nuclear linkage whereas gravitation forces at 10⁻⁴⁰ seems minuscule compared to the strong nuclear linkage. Nevertheless, we know that gravitational force is responsible for apples falling from trees and for the orbit of planets.

Of the elementary forces, electromagnetic force is of particular interest in medicine since all biochemical properties are controlled by it. Electromagnetic forces are transmitted by photons. In contrast to gluons, which have no mass and can only move in femto-distance (10^{31 15} meters) within the atom, photons are able to fly nearly without any limitation.

Oscillation is a force that appears in a certain periodicity recurring at specific distances in time. Oscillations (waves, rays) for transmission can be coupled with matter particles or not. Sound waves, electrical alternating currents (AC), and earthquake waves are typical representatives which need matter as a medium to be able to spread. In contrast light, heat, and electromagnetic fields are independent of any matter particles as spreading medium. From the point of view of a receiver, the properties of an oscillation are: frequency, intensity, wave property, polarization, and coherence.

To be able to observe resonance (reverberation) an oscillation must hit a system able to resonate (resonator). Thus an essential property of a resonator is its ability to store oscillation energy. If the frequency, phase, and intensity are fitting, the resonator is stimulated and an exchange of energy takes place. If energy is being provided constantly the stimulation increases up to its natural limit under attenuation. The oscillation stimulated as a result in the resonator can be many times bigger than the oscillation fed into it.

Bioresonance therapy assumes as a basic concept of thought that a super ordinate (dominant) energy field with electromagnetic properties exists. This super ordinate energy field is situated in and around the organism and decreases with the square of distance—as is the same in all electromagnetic fields. Thus, biochemical processes are subordinated to this control and regulation level. So before changes through strains or illnesses can take place on the level of the body there obligatory has to be changes in the electromagnetic status. Within an organism, there are several control mechanisms. The cells have control, there is control of the tissues, there is control of systems (such as the nervous system and the hormonal system), and electromagnetic control and regulation. Therefore, in the body, there are different relevant therapeutic levels: electromagnetic, electrical, humeral, biochemical, and mechanical levels. Not only do organisms have controlling electromagnetic energy fields, all substances define their chemical and physical properties through electromagnetic pre-requisites. This is the reason why the electromagnetic fields of external substances are used in a calculated way in bioresonance therapy, for example, to reduce intolerance to specific allergens or strains in organisms.

Every substance has a frequency spectrum of electromagnetic waves specific to it that is comparable to an electromagnetic fingerprint. This frequency spectrum is characteristic for a substance and is used to identify substances in several areas (i.e. infrared spectroscopy). For example, with the magnet resonance imaging (MRI) the above mentioned matter can be determined over the magnet resonance frequencies.

For most organic molecules or organic systems the highest intensity of these electromagnetic rays is in the infrared frequency area. Thus a human being in this frequency range radiates approximately with the intensity of a 100 w light bulb. In other words, an atom on the surface of a human being radiates approximately 2000 photons per second just in the infrared frequency band alone.

In some embodiments of the present invention bioresonance therapy uses the therapeutically much more effective extreme low frequency range between 10 Hz and 150 KHz. Because the photon energy in this area is extremely weak these signals are called ultra weak or ultra fine. However, there is not always a correlation between strength and effectiveness of signals. This is particularly true in living organisms because their systems are non-linear. Also in the frequency range used by bioresonance therapy every cell has its own particular electromagnetic field. Cells of identical structure have identical fields. Cells differing in structure have differing fields. Thus cell structure, organs and body systems can show electromagnetic resonances. In healthy people the field specific to cells vary very little, if at all.

Resonance frequencies are those ranges (spectrums) in which energy is best transmitted and thus in which the therapeutic effect is the biggest. The body overall consists of a gigantic mix of frequencies organism from the electromagnetic point of view. Many of the resonance frequencies of organs and body systems have been assessed by bioresonance therapy and can be addressed therapeutically in an aimed way.

Control oscillations are necessary for the control of physiologic processes. Exogenous signals are needed to stimulate control oscillations because an organism is an open system. Apart from mechanical impulses such as movement and chemical impulses such as food, water and oxygen, the body needs environmental stimuli in the form of light, sound, Schumann waves, gravitation, etc. If these exogenous impulses are missing, as a consequence this will lead to degenerative processes caused by the decrease of intensity of control oscillations.

Apart from the physiological, electromagnetic oscillation in every human being, there also are pathological oscillations, or so-called “interference oscillation”. Depending on the intensity of the interference oscillation and the sensitivity on the part of the organism these interference oscillations interfere with physiological processes. Essentially interference oscillations are caused by radiation strains, toxins, microorganisms that are pathogenic to humans, psychological effects, physical damage, focal strains and allergies or other intolerances.

Together, the control and interference oscillation can be referred to as the “Patient's Own Oscillation” (POO). The control and interference oscillation of an organism can be measured through electrodes without straining the organism in any way. In this process the electrodes act as aerials, in other words, they convert the electromagnetic fields into tension potentials. These tension potentials can be led to the input of a bioresonance device by cables. As with magnetic resonance, there are different forms of electrodes that are suited for measuring different parts and functions of the body.

The control and interference oscillation measured in an organism can be processed for therapeutic purposes by high-quality electronics that are specially developed for the processing of these ultra weak signals to insure accurate measurement over the total frequency range. Depending upon the organ resonance system chosen and the individual characteristics of the patient, different amplification and attenuation steps of treatment are available. Amplification or attenuation in itself does not change the highly complex oscillations measured from the organism in its wave properties, only the intensity changes. To use biotherapeutic resonance with the optimal transfer of energy and thus stimulation it is absolutely necessary to determine the proper amplifications or attenuations. To stimulate different structures of differing sizes, such as organs versus body systems, the size and structure must be taken into account. If too high a force is used and an over-taxation of the organ or system takes place, no stimulation occurs. Each organ or body system is unique and the individual resonance must be determined for maximal effectiveness.

One aim of the therapy in some embodiments of the present invention is the support and reinforcement of physiologic processes (control oscillation). The other aim of the therapy is to reduce and, if possible, eliminate the strains pathologic to humans (interference oscillation). These two different and complementary goals of therapy that can be realized by bioresonance therapy.

An aim of the therapy in some embodiments of the present invention is to give the organism a therapy signal as exactly fitting as possible. The more accurate the therapy signal the bigger and faster the resonance and therefore the therapeutic effect.

In is believed that the success achieved in bioresonance therapy may be due in part to the basis for the therapy signal stems from every patient individually. Thus we have a very precise energetic image as the basis for therapy.

If waves are superseded (superposition), this is generally called interference. The process to eliminate interference oscillation in the bioresonance therapy is called destructive interference. An interference signal is taken from the body and is reversed in its polarity, inversed and returned to the organism. Plus becomes minus and minus becomes plus. The overlapping of the exactly identical but opposite oscillations creates the phenomenon of destructive interference in the organism. In this case destructive interference signals are reduced or even completely eliminated in their effect. If a reduction or elimination of interference signals takes place, it is turned into heat in the organism (law of preservation of energy), and the interference signals no longer exist.

As mentioned above, before interference signal are emitted (radiated) by, for instance, toxins. The reduction of these pathologic oscillations has as a consequence that certain electromagnetic properties of pathogenic substances are changed (for example, the linkage force of the toxins). When the linkage forces are changed, the organism has the opportunity to break down such substances. As a result, the patient experiences increased drainage of the toxin. As a result of the increased drainage, patients are instructed to drink sufficient water low in minerals to assist in the clearance of the toxins after the therapy.

But it is not only the stored pathogen substances themselves that interfere with the body. The interference signals emitted by the pathogens also have a negative influence on the interstitium. The interference oscillations are stored in the H-bridges of systematic water structures, the H₂O clusters. The consequence of the pathologic oscillations being stored there is a marked decrease of the environment and a concomitant decrease in supplies to the cells. The interference oscillations stored in the interstitium can also be reduced and possibly completely eliminated by bioresonance therapy in embodiments of the present invention.

All patients have an individual reaction time to therapies. Some are able to react quicker than others. Surely many complex factors contribute to this: constitution, strain, psychological situation, age, etc. Bioresonance therapy takes these circumstances into account by optimizing techniques of the so-called cybernetic control circuits.

During the therapy the patient's own oscillations are measured permanently and processed for therapy by selection of frequency, amplification, inversion, for example. If a complex pathologic oscillation in the organism changes due to the influence of the therapy then immediately the therapy signal changes as well because it's source is the body itself.

Control and interference oscillations supersede the biochemical processes of the organism. The complete spectrum of all oscillations is measured through special electrode (Aerial effect). By selecting the frequency depending on the selection organs or body systems, which have different resonance frequencies on the basis of their structure, particular problems in these organs or systems can be addressed.

By a further separation in the pre selected frequency range of control and interference oscillation it is possible on the one hand to reduce the interference oscillation by destructive interference (counter oscillation) and on the other hand to reinforce the control oscillation by supper-sedation (constructive interference). This is obtained with the necessary amplification-attenuation settings, which depends upon the organs or body system, respectively, to be treated and the general regulation properties of the patient.

As will be realized from the foregoing discussion, a key to understand bioresonance therapy lies in knowing and understanding the fact that all vital processes in the organism are influenced and controlled by electro-magnetic oscillation. The electromagnetic processes are functionally super ordinates to biochemical processes. Matter is manifestation of energy. Biochemical processes in the organism always have a component in the domain of the photons. Therefore, it can be seen that the organism, at this level, supplies information, which is not available chemically, but can be determined by electronic means. From this point of view, disease, the disturbance of the biochemical sequences in biological systems, can be regarded as disturbance to the order of electromagnetic oscillation in the body, triggered by both exogenous and endogenous influences.

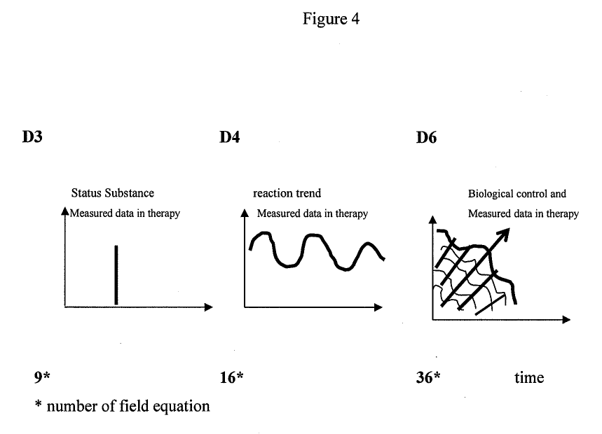

Some embodiments of the present invention may rely on diagnosis in multiple dimensions as represented in FIG. 4. D3 illustrates the minimal amount of information in the usual measurements of only one status (e.g. blood status) at a particular time (e.g. in the morning), 3×3 (=9). By D4, additional account is taken of time (e.g. exercise ECG glucose tolerance test). In this way the quantity of information is increased to 4×4 (=16) equations. The method of holistic medicine with all six dimensions, which produce more items of information 6×6 (=36) i.e. four times more than in the pure status picture D3. Further statements on the morphogenetic field of the patient are thus obtained.

### EXAMPLES

The invention will be illustrated in further detail with the following Examples.

### Example 1

An example of a treatment protocol including some embodiments of the present is described with reference to diagnosis, initial treatment, and follow-on treatment. The patient will have been pre-screened to determine, for example, the absence of a pacemaker, implanted medical devices, transplanted organs, and/or other conditions that would preclude the use of bioresonance therapy.

Diagnosis:

The patient's weight, temperature, blood sugar, blood pressure, and heart rate are recorded at the beginning of every session as a routine procedure. If the patient is diabetic, his blood sugar is recorded too. Then, the frequency of the patient's organs is measured through certain points in both hands and feet. A total number of 55 points is measured. The measured organs and systems include: lymphatic system, lungs, colon, nervous system, circulatory system, heart, glands, small intestine, spleen, pancreas, liver, joints, stomach, connective tissues, skin, fat, gall bladder, kidney, bladder, prostate/ovaries, right and left hemispheres.

After the initial measurements, a drop of blood is then taken from the patient and the frequency of the organs is measured again. This will detect whether the organs are normal or involved in acute or chronic diseases. By measuring the frequency of the lymphatic system, blood frequency can be determined to be normal or defective. Blood frequency is an indicator that determines the general health status of the body. When it is defective, then this is a good sign that indicates a disturbance in the body.

A special painless test (identification test) is then performed to detect the frequency of environmental, industrial, food and other substances that could cause irritation to the patient. The substances to be tested are chosen according to the patient's complaint. The various test kits may include: 220 industrial substances, 35 heavy metals, 40 substances of pollen-related allergens, drugs, 5 vaccines, 33 tumor samples, 200 food allergens, 60 parasites, the patient's own excrements (urine, stool, saliva), or other, external substances (make up, perfume, shampoo, etc.).

Treatment:

The treatment plan is set according to the diagnostic findings. All patients are first subjected to a basic program and then according to their complaints they receive the appropriate programs they need. The basic program manages the following: eliminating all the blocks that could slow the process of recovery such as: abnormal blood frequency, presence of heavy metals resulting from polluted surroundings, non-appropriate foods, medications, bacteria, fungi, other microorganisms, and vitamin deficiencies or excesses. The basic treatment program also addresses the functions of: enhancing communication between the right and left hemispheres, improving communication between the brain and organs, enhancing communication among all organs of the body, enhancing the detoxification process in the body, activating the immune system, enhancing metabolic processes though liver activation, managing heavy metals, as well as eliminating bacteria, fungi, and parasites.

The following programs vary from one patient to another. Different programs which treat pain, allergy, parasitic infestation, and other concerns are then given to the patient. After treatment, a bottle (50 mL) of normal saline is given to the patient and administered for its use. All patients are asked to have 3 drops of the normal saline in one glass of water twice a day. They are asked to keep the bottle away from sunlight, neon, halogen, and tungsten lamps, and away from the mobile phone and refrigerator. Every time the patient sits for a session he gets a new bottle.

Follow-up Sessions:

Follow-up sessions vary according to the patient's status. Each session of the patient is compared to the previous measurement and with next one, to compare the frequency of the organs as well the substances and the blocks in the body.

New cases with severe complaints are usually given an appointment once a week. Once they start recovering they sit for a session every two weeks. As they restore their normal frequencies, they are advised to sit for a session every one to two months.

### Example 2

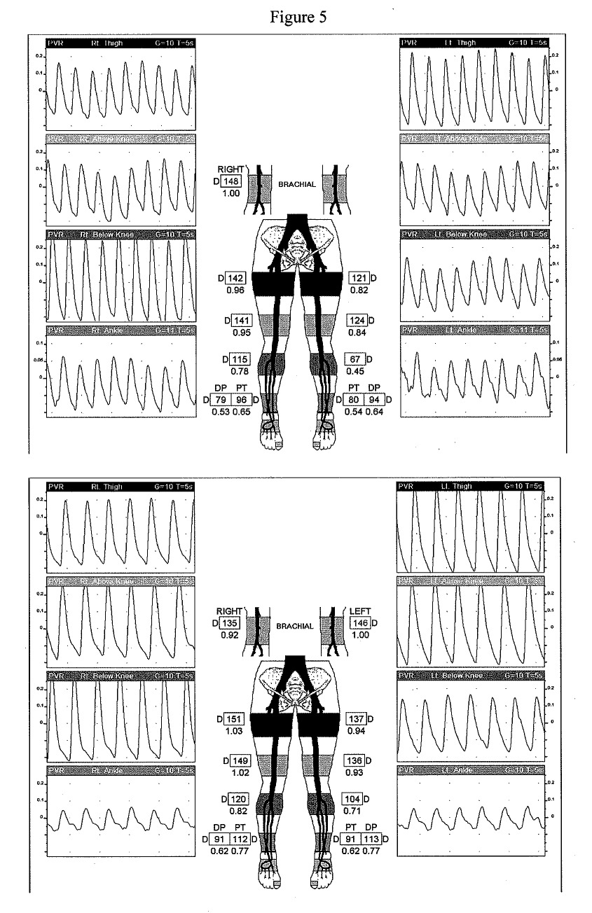

Bioresonance therapy can be used to treat peripheral vascular disease in diabetic patients. The Ankle Brachial Pressure Index (ABPI) is a measure of the fall in blood pressure in the arteries supplying the legs. ABPI is frequently used to detect evidence of blockages. To determine ABPI, the highest systolic blood pressure in the arteries at the ankle is divided by the highest of the two systolic blood pressures measured in the arms. A patient's organ frequencies are measured based upon the ABPI measurements. The range of frequencies used are between 1.9 and 3.9 Hz. After the correct frequency is determined, a patient has 7-10 sessions over the course of two months. At each session, the ABPI and the organ frequencies will be calculated and then compared between sessions. FIG. 5 is representative of the differences in a patient's ABPI before and after receiving bioresonance therapy.

### Example 3

Bioresonance therapy is an effective method to rid the body of parasitic infections. Parasitic infections have been linked the incidence of some forms of cancer or other chronic diseases. Bioresonance therapy was used to detect parasitic infection in the blood of chronic disease patients, such as those presenting with allergic diseases or chronic joint pain. These patients were treated with bioresonance therapy that eliminated the parasite causing disease.

The behavior of parasites in the human body changes the parasite's entire ecosystem. Parasites thrive on the nutrition that is best for the host, generally animal protein. Parasites penetrate the cell membranes of red blood cells and induce damage to the organ infested. The infestation will result in general symptoms such as joint and muscle aches, allergies, anemia, drowsiness, nervousness, immune dysfunction, chronic fatigue, sleep disturbances, and diarrhea.

Bioresonance therapy is used to treat parasitic infection by detecting the location of the parasite, measuring its intensity, eliminating the parasite, and then regenerating the organ that was infested. Samples used to detect the parasite can be detected in the blood as well as in the measured organ. Some of the parasites are known species, while others are not yet identified to be related to any relevant species. The frequency of the human body is changeable, therefore similarities between the frequencies of the parasite and other endogenous substances may occur.

The treatment of parasitic infections includes subjecting the patient to several therapy programs that enhance the function of the immune system, the detoxification process of the liver, and mucus secretion (defense barrier) in the colon. To accelerate the process of parasite elimination, patients are put on a diet free of animal proteins, the major source of nutrients for parasites, for at least one month.

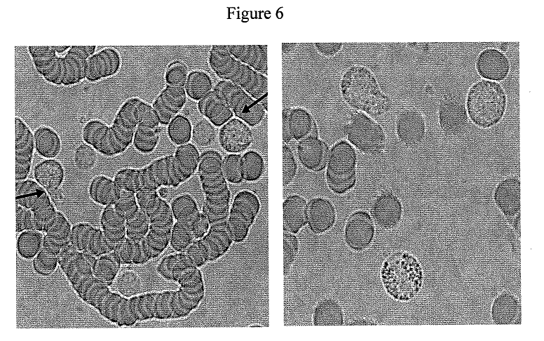

The blood film of a hypothyroidism patient is presented in FIG. 6. As is illustrated by the figure, the parasite infection has been eliminated and the immune system has been stimulated, as evidenced by the increased size of the white blood cells.

Those of ordinary skill in the art will appreciate that the inventive concepts described herein are applicable to a number of embodiments that should be considered within the scope of the present invention. Accordingly, the description herein should not be read as limiting the present invention, as other embodiments also fall within the scope of the present invention.

We claim:

1. A method for diagnosing a condition in a patient, the method comprising the steps of:
a) exposing at least a portion of a patient to an external field;
b) determining a bioresonance; and
c) correlating the bioresonance with the bioresonance of a disease state or symptom.

2. The method of claim 1, wherein the disease state is diabetes.

3. The method of claim 1, wherein the disease state is cancer.

4. The method of claim 1, wherein the disease is a gynecological disease.

5. The method of claim 1, wherein the disease state is chronic.

6. The method of claim 5, wherein the chronic disease is eczema.

7. A method for the treatment of pain, suffering or discomfort in an individual, the method comprising the steps of:
a) exposing at least a portion of an individual to an external field; and
b) affecting the bioresonance of the portion of the individual that was exposed to the external field;
in a way that alleviates the pain, suffering or discomfort of the individual.

8. A method for diagnosing an infection in a patient, the method comprising the steps of:
a) exposing at least a portion of a patient to an external field;
b) determining a bioresonance; and
c) correlating the bioresonance with a specific location of infection.

9. The method of claim 8, wherein the infection is a parasitic infection.

10. The method of claim 9, wherein the parasitic infection is a localized infection.

11. The method of claim 9, wherein the parasitic infection is a systemic infection.

12. A method for the treatment of an infection, the method comprising the steps of:
a) exposing at least a portion of the individual to an external field;
b) affecting the bioresonance of a portion of the individual that was exposed to the external field;
in a way that eradicates the infection.

13. The method of claim 12, comprising the additional step of:
c) having the individual follow a diet free of animal proteins.

14. The method of claim 12, wherein the infection is a parasitic infection.
